(19) United States
(12) Patent Application Publication
(10) Pub. No.: US 2006/0094762 A1
(43) Pub. Date: May 4, 2006

(54) Magnesium salt of imidazole derivative

(76) Inventors: Yatendra Kumar, Gurgaon, Haryana (IN); Mohan Prasad, Gurgaon, Haryana (IN); Neela Praveen Kumar, Hyderabad, Andhra (IN)

Correspondence Address: RANBAXY INC., 600 COLLEGE ROAD EAST, SUITE 2100, PRINCETON, NJ, 08540, US

(21) Appl. No.: 10/541,140

(22) PCT Filed: Jan. 7, 2004
(86) PCT No.: PCT/IB04/00010

§ 371 Date: Jun. 30, 2005

(30) Foreign Application Priority Data
Jan. 7, 2003 (IN) 20DEL2003

(51) Int. Cl. A61K 31/44

(52) U.S. Cl. 514/338

(57) ABSTRACT

Magnesium salts of rabeprazole, processes for preparing them, pharmaceutical compositions of the salts and their use in treatment or prevention of gastrointestinal ulcers are provided.

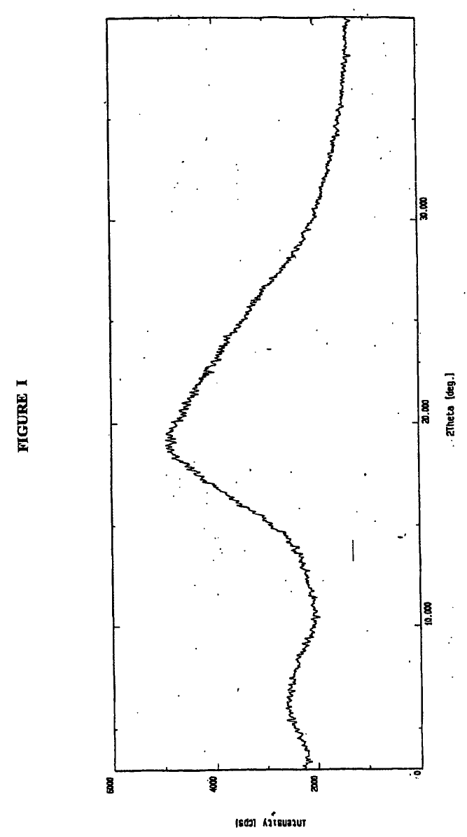

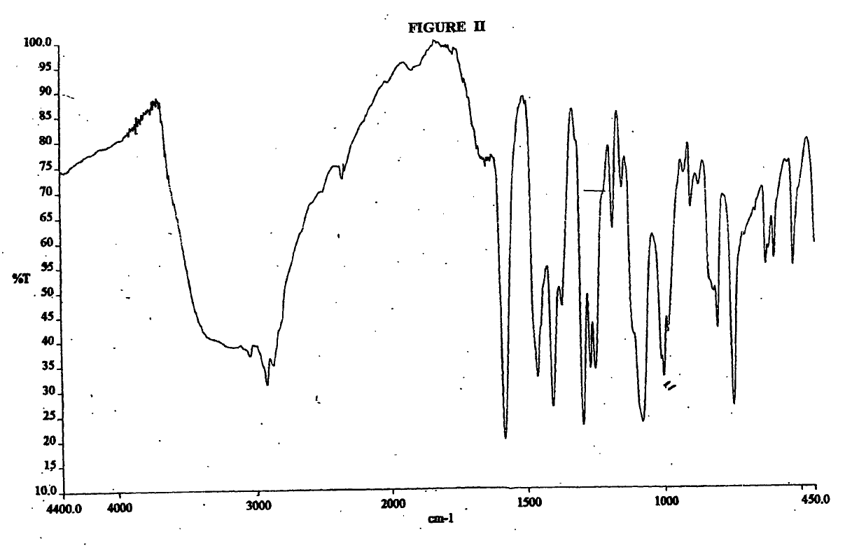

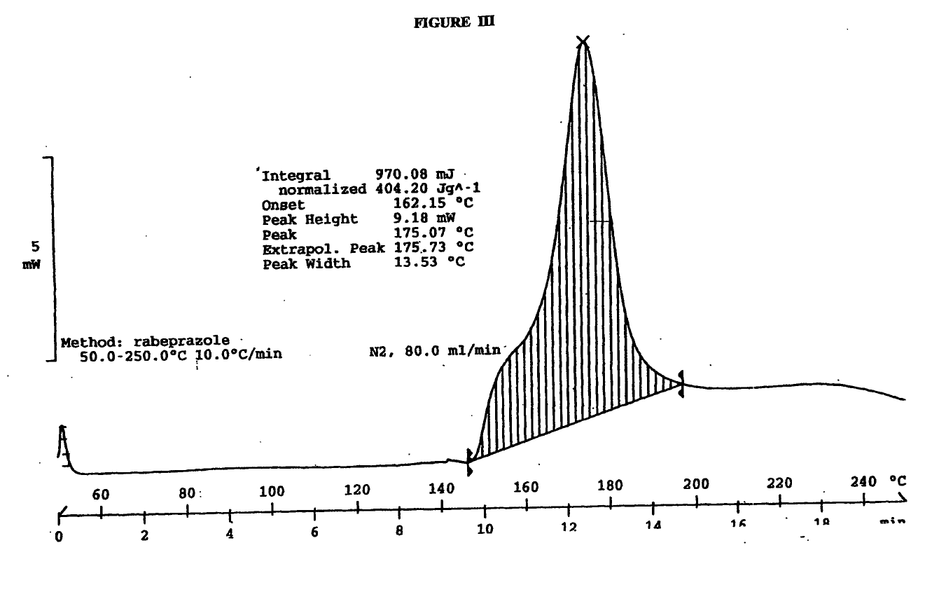

### FIELD OF THE INVENTION

Magnesium salts of rabeprazole, processes for preparing them, pharmaceutical compositions of the salts and their use in treatment or prevention of gastrointestinal ulcers are provided.

### BACKGROUND OF THE INVENTION

U.S. Pat. No. 5,045,552 discloses several substituted pyridylmethylsulfinyl benzimidazoles, including 2-[[4-(3-methoxypropoxy)-3-methyl-2-pyridinyl]-methylsulfinyl]-1H-benzimidazole, i.e., rabeprazole. Rabeprazole is a proton-pump inhibitor and an antibacterial agent. Rabeprazole sodium is used for treating and preventing peptic ulcers, and for treating bacterial infections caused by camphylobacter and helicobacter pylori.

While this patent mentions that some of the disclosed compounds can form a salt with metals such as sodium, potassium, calcium or magnesium, only sodium salts of the disclosed compounds have been prepared. In particular, only the sodium salt of rabeprazole has been synthesized besides rabeprazole. Also, the commercial product of rabeprazole (Aciphex by Eisai Inc.) uses rabeprazole sodium. Rabeprazole sodium is obtained in amorphous form by the process described in this patent, and is hygroscopic in nature.

A recent Japanese Patent Application JP 2001 039975 describes nonhygroscopic crystals of benzimidazolyl pyridylmethyl sulfoxides, including rabeprazole sodium, and their preparation.

### SUMMARY OF THE INVENTION

Magnesium salts of rabeprazole, i.e., rabeprazole magnesium are provided. A particular form of rabeprazole magnesium is rabeprazole hemimagnesium. Another aspect relates to rabeprazole magnesium in an amorphous form.

Processes for preparing rabeprazole magnesium are provided, which comprise contacting rabeprazole or its sodium salt with the magnesium salt of an acid in a solvent to form rabeprazole magnesium, wherein the process is carried out in the presence of a base whenever rabeprazole is used.

Alternative processes for preparing rabeprazole magnesium are also provided, which comprise reacting rabeprazole with magnesium alkoxide in an alcohol as solvent to form rabeprazole magnesium.

Further aspects include methods for treating or preventing gastrointestinal ulcers which comprises administering to a patient in need thereof an effective amount of rabeprazole magnesium, and a pharmaceutical composition for use in the treatment or prevention of gastrointestinal ulcers that comprises an effective amount of rabeprazole magnesium along with pharmaceutically acceptable excipients.

### DETAILED DESCRIPTION OF THE INVENTION

The term “rabeprazole magnesium” as used herein means any comprised of rabeprazole anions and magnesium cations. For instance, solid as well as dissolved forms are included, as are various crystalline and amorphous forms.

Further, the term “rabeprazole magnesium” encompasses stoichiometric as well as non-stoichiometric ratios of rabeprazole anion and magnesium cation. The ratio of rabeprazole to magnesium is not required to be 1:1 in order to be termed rabeprazole magnesium. Rabeprazole magnesium is preferably formed as a salt having a 2:1 molar ratio between rabeprazole anion and magnesium cation (i.e., rabeprazole hemimagnesium). Rabeprazole hemimagnesium may be formed even when an excess of rabeprazole or an excess of magnesium salt of an acid is used in the salt formation.

Rabeprazole magnesium obtained in an amorphous forms is non hygroscopic. The amorphous form may be advantageous in comparison with the crystalline form as it is obtained as a finely powdered material with better solubility properties.

Rabeprazole magnesium, and particularly rabeprazole hemimagnesium, may exist in an anhydrous and/or solvent-free form or as a hydrate and/or a solvate. The hydrates of rabeprazole magnesium, especially hydrates of rabeprazole hemimagnesium provide aspects further.

Further, rabeprazole magnesium can exist as one of two enantiomers due to the presence of a chiral center. The enantiomers may either be separated, e.g., by subjecting rabeprazole or the sodium salt to resolution using an optical purity embedding agent (for example, as described in CN 1,223,262, see Chem. Abs. 133:17460) and converted to the corresponding magnesium salt, or prepared by stereo-selective oxidation of the corresponding sulfide in the presence of a chiral titanium complex and a base (U.S. Pat. No. 5,948,789), and converted to the corresponding magnesium salt The individual enantiomers as well as mixtures thereof are likewise all embraced by the expression “rabeprazole magnesium.”

Rabeprazole magnesium can be prepared by processes comprising contacting rabeprazole or its sodium salt, with magnesium salt of an acid in a suitable solvent, preferably with both the magnesium salt of an acid and rabeprazole or its sodium salt being fully dissolved therein.

Whenever rabeprazole is used, the process is carried out in the presence of a base. Suitable bases which may be used in the process along with rabeprazole include alkali metal hydroxides such as sodium hydroxide or potassium hydroxide, alkali metal carbonates such as sodium carbonate or potassium carbonate, and alkali metal bicarbonates such as sodium bicarbonate.

Magnesium salt of an acid to be used in the process can be the salt of any inorganic or organic acid such as, magnesium chloride, magnesium nitrate, magnesium sulphate, magnesium phosphate, magnesium carbonate, magnesium dihydrogenphosphate, magnesium oxalate, magnesium acetate, magnesium lactate, magnesium succinate, magnesium citrate, and magnesium tartrate.

Suitable solvents for carrying out the process include water, alcohols such as methanol, ethanol or isopropanol, ketones such as acetone or methyl isobutyl ketone, esters such as ethyl acetate, ethers such as dioxan or tetrahydrofuran, nitrites such as acetonitrile, dipolar aprotic solvents such as dimethylsulfoxide or dimethylformamide, hydrocarbons such as hexane or toluene and mixtures thereof.

Water is one particular solvent. The reactants are generally more soluble than the rabeprazole magnesium product. In this way, the salt forming reaction can be accompanied by spontaneous precipitation of the produced magnesium salt out of the solution. The above described methods allow for the production of rabeprazole magnesium in an amorphous form.

Alternatively, the precipitation may be facilitated by reducing the volume of the solution and/or by adding an antisolvent, i.e. a solvent in which the rabeprazole magnesium is insoluble or sparingly soluble. The precipitation can also be induced by reducing the temperature of the solvent, especially if the initial temperature at contact is elevated. Crystalline or partially crystalline material may sometimes be obtained by such processes.

Rabeprazole magnesium can be prepared by an alternative process, which comprises reacting rabeprazole with magnesium alkoxide in an alcohol as solvent to form rabeprazole magnesium. Commercially available magnesium alkoxide may be used, or it may be generated in situ by refluxing magnesium metal (turnings or ribbon) in the corresponding alcohol, which also acts as a solvent.

Suitable alkoxides which may be used in the process include magnesium methoxide, magnesium ethoxide, magnesium propoxide and magnesium isopropoxide. Suitable alcohols include methanol, ethanol, propanol and isopropanol.

The rabeprazole or its sodium salt to be used in the preparation processes can be obtained by methods known in the art, including those described in the above mentioned patent, as well as in U.S. Pat. No. 6,313,303, and the international patent applications WO 01/04109, WO 02/062786 and WO 02/083608.

Generally, the rabeprazole magnesium is precipitated out of the solution or reaction mixture. The precipitation may be facilitated by reducing the volume of the solution and/or by adding an antisolvent, i.e., a solvent in which the rabeprazole magnesium is insoluble or sparingly soluble. The precipitation can also be induced by reducing the temperature of the solvent, especially if the initial temperature at contact is elevated.

The precipitated magnesium salt may be isolated in a solid state by conventional methods such as filtration or centrifugation, optionally followed by washing and/or drying and may be purified by crystallization.

Rabeprazole magnesium is a useful proton-pump inhibitor and an antibacterial agent, and thus can be used to treat any condition that would be benefited by administration of a gastric acid secretion inhibitor. In particular, rabeprazole magnesium can be used for healing of erosive or ulcerative gastroesophageal reflux disease (GERD); maintenance of healing of erosive or ulcerative GERD; healing of duodenal ulcer; treatment of pathological hypersecretory conditions, including Zollinger-Ellison Syndrome, by administering an effective amount of the salt to a patient in need thereof. The specific form of rabeprazole magnesium to be used is not particularly limited and specifically includes rabeprazole hemimagnesium.

The salt can be administered by any suitable route including oral, parenteral or transdermal. The “patients” intended to be treated include human and non-human mammals.

The salt is usually administered as part of a pharmaceutical composition. Accordingly, a further aspect of the invention is a pharmaceutical composition for treating or preventing gastrointestinal ulcers that comprises an effective amount of rabeprazole magnesium and pharmaceutically acceptable excipients. The salt may be conveniently formulated into tablets, suspensions, injectables and other pharmaceutical forms.

In the following section, particular embodiments are described by way of examples to illustrate the processes described herein. However, these are not intended in any way to limit the scope of the present invention. Variants of these examples would be evident to persons ordinarily skilled in the art.

### EXAMPLE 1

### 2-[[4-(3-methoxypropoxy)-3-methyl-2-pyridinyl]-methylsulfinyl]-1H-benzimidazole, hemimagnesium salt

Magnesium turnings (11.0 g, 0.004 m) were refluxed in methanol (100 ml) with a speck of iodine for two hours. The above solution was cooled to 20° C. and rabeprazole base (25 g, 0.0696 m) was added. The mixture was stirred for 15 minutes and the solution filtered to remove any undissolved material. The filtrate was concentrated under reduced pressure. The residue was stirred in ethyl acetate (100 ml) for 30 minutes. The solid obtained was filtered, washed with ethyl acetate and dried at 40° C. under vacuum to give rabeprazole magnesium (21.9 g).

Assay (by HPLC): 98.8%, Water (w/w): 6.88%, Mg content (w/w): 2.96%

¹HMR (CDCl₃, δ, ppm); 2.03-2.04(m, 5H), 3.33(s, 3H), 3.48-3.52(t, 2H), 4.02 (t, 2H), 4.72(bs, 2H), 6.63(bs, 1H), 7.25(d, 2H), 7.57(m, 2H), 8.22(d, 1H).

XRD, IR and DSC spectra are as shown in FIG. I II, & III respectively.

### EXAMPLE 2

### 2-[[4-(3-methoxypropoxy)-3-methyl-2-pyridinyl]-methylsulfinyl]-1H-benzimidazole, hemimagnesium salt

Rabeprazole sodium (10 g, 0.0262 m) was dissolved in methanol (175 ml) at room temperature. Magnesium acetate (2.8 g, 0.013 m) was added to the above solution. The solution was stirred for 1 hour at room temperature, and then filtered to remove the undissolved particles. The filtrate was concentrated under reduced pressure. The residue was stirred in diisopropyl ether 30 minutes, the solid obtained was filtered, and dried. It was then suspended in water (50 ml), stirred for 30 minutes, filtered, washed with water and dried at 40° C. under vacuum to give 8.5 g of white partially crystalline rabeprazole magnesium. Water (w/w): 6.11%.

### EXAMPLE 3

### 2-[[4-(3-methoxypropoxy)-3-methyl-2-pyridinyl]-methylsulfinyl]-1H-benzimidazole, hemimagnesium salt

Rabeprazole sodium (10 g, 0.0262 m) was dissolved in water (75 ml) at room temperature. Magnesium acetate (2.8 g, 0.013 m) dissolved in water (25 ml) was slowly added to the above solution in 30 minutes. Rabeprazole magnesium precipitated out simultaneously. The suspension was further stirred for 30 minutes, the obtained solid was filtered and washed with water. The product was dried at 40° C. under reduced pressure to give rabeprazole magnesium (8.8 g).

Water (w/w): 5.8%; XRD, IR and DSC spectra are similar to those for example 1.

### EXAMPLE 4

### 2-[[(3-methoxypropoxy)-3-methyl-2-pyridinyl]-methylsulfinyl]-1H-benzimidazole, hemimagnesium salt

Sodium hydroxide flakes (1.1 g, 0.027 m) were dissolved in water (80 ml). To the above solution was added rabeprazole base (10 g, 0.027 m) at 10-15° C. and stirred till a clear solution was obtained. Magnesium acetate (3.0 g, 0.027 m) dissolved in water (20 ml) was added to the resulting solution. The reaction mixture was stirred for 30 minutes. The solid that separated out was filtered, washed with water and dried under vacuum at 40° C. to give rabeprazole magnesium (8.8 g).

Water (w/w): 5.46%; XRD, IR and DSC spectra are similar to those for example 1.

We claim:

1. A magnesium salt of rabeprazole.

2. The salt according to claim 1, which is rabeprazole hemimagnesium.

3. The salt according to claim 1 or 2, which is in a hydrated form.

4. The salt according to claim 1 or 2, which is in an amorphous form.

5. A process for preparing rabeprazole magnesium, which comprises contacting rabeprazole or its sodium salt with magnesium salt of an acid in a solvent, to form rabeprazole magnesium, wherein the process is carried out in the presence of a base when rabeprazole is used.

6. The process according to claim 5, wherein the magnesium salt of an inorganic acid is used.

7. The process according to claim 6, wherein the magnesium salt is selected from magnesium chloride, magnesium nitrate, magnesium sulphate, magnesium, phosphate, magnesium carbonate, or magnesium dihydrogenphosphate.

8. The process according to claim 5, wherein the magnesium salt of an organic acid is used.

9. The process according to claim 8, wherein the magnesium salt is selected from magnesium oxalate, magnesium acetate, magnesium lactate, magnesium succinate, magnesium citrate, or magnesium tartrate.

10. The process according to claim 5, wherein the base is selected from alkali metal hydroxides, alkali metal carbonates, or alkali metal bicarbonates.

11. The process according to claim 10, wherein the base is selected from sodium hydroxide, potassium hydroxide, sodium carbonate, potassium carbonate, or sodium bicarbonate.

12. The process according to claims 5, wherein the solvent is selected from water, alcohol, ketone, ester, ether, nitrile, dipolar aprotic solvent, or hydrocarbon or mixtures thereof.

13. The process according to claim 12, wherein the solvent is selected from methanol, ethanol, propanol, acetone, methyl isobutyl ketone, ethylacetate, dioxan, tetrahydrofuran, acetonitrile, dimethylsulfoxide, dimethylformamide, hexane, or toluene or mixtures thereof.

14. The process according to claim 5, wherein rabeprazole magnesium precipitates spontaneously from the solvent.

15. A process for preparing rabeprazole magnesium, which comprises reacting rabeprazole with magnesium alkoxide in an alcohol.

16. The process according to claim 15, wherein magnesium alkoxide is generated in situ by refluxing magnesium metal in the corresponding alcohol.

17. The process according to claim 15, wherein magnesium alkoxide is selected from magnesium methoxide, magnesium ethoxide, magnesium propoxide and magnesium isopropoxide.

18. The process according to claim 15, wherein the alcohol is selected from methanol ethanol, propanol or isopropanol.

19. The process according to claim 5 or 15, wherein rabeprazole hemimagnesium is formed.

20. The process according to claim 5, 15 or 19, wherein a hydrated form of rabeprazole magnesium is obtained.

21. The process according to claim 5, 15 or 19, wherein an amorphous form of rabeprazole magnesium is obtained.

22. A method for treating or preventing gastrointestinal ulcers, which comprises administering to a patient in need thereof an effective amount of rabeprazole magnesium.

23. The method according to claim 22, wherein rabeprazole magnesium is used for healing of erosive or ulcerative gastroesophageal reflux disease (GERD); maintenance of healing of erosive or ulcerative GERD; healing of duodenal ulcer; or treatment of pathological hypersecretory conditions, including Zollinger-Ellison Syndrome.

24. The method according to claim 22, or 23 wherein rabeprazole hemimagnesium is administered.

25. A pharmaceutical composition for use in the treatment or prevention of gastrointestinal ulcers comprising an effective amount of rabeprazole magnesium and pharmaceutically acceptable excipients.

26. The pharmaceutical composition according to claim 25, wherein rabeprazole hemimagnesium is used.

27. The pharmaceutical composition according to claim 25, or 26 wherein a hydrate of rabeprazole magnesium is used.

28. The pharmaceutical composition according to claim 25, or 26 wherein an amorphous form of rabeprazole magnesium is used.
